(19) United States
(12) Patent Application Publication
(10) Pub. No.: US 2024/0315764 A1
(43) Pub. Date: Sep. 26, 2024

(54) RESECTOSCOPE

(71) Applicant: OLYMPUS WINTER & IBE GMBH, Hamburg (DE)

(72) Inventors: Christian BROCKMANN, Hollenstedt (DE); Andreas OFFT, Reinbek (DE); Katica MAUCH, Schönefeld (DE)

(73) Assignee: OLYMPUS WINTER & IBE GMBH, Hamburg (DE)

(21) Appl. No.: 18/407,037

(22) Filed: Jan. 8, 2024

Related U.S. Application Data
(60) Provisional application No. 63/454,160, filed on Mar. 23, 2023.

(51) Int. Cl. A61B 18/14; A61B 18/00

(52) U.S. Cl. CPC A61B 18/1485; A61B 2018/00083; A61B 2018/00166; A61B 2018/00964; A61B 2018/00982; A61B 2218/002

(57) ABSTRACT

A resectoscope in which the volume flows for the flushing fluid are optimized and at the same time a particularly laminar flow is formed in front of the distal end of the resectoscope in order to improve the visibility during treatment. This is achieved by the fact that the resectoscope has a shaft with an outer tube and an inner tube. The inner tube serves as an inflow, and a volume between the outer tube and the interior serves as an outflow for a flushing fluid. The inner tube has different cross sections at its distal and proximal regions.

The invention relates to a resectoscope according to the preamble of claim 1.

Resectoscopes of the type in question are mainly used for endoscopic applications in urology and gynecology, where they are preferably used for treatment in the region of the bladder or prostate. However, the field of application of these medical instruments is not limited to these regions and instead includes the treatment of all organs, preferably of the human abdomen.

In a standard design, resectoscopes of the type in question have a working element. For the treatment of the diseased organs, the resectoscope, which usually has an elongate shaft formed by an outer tube, is inserted through an opening into the patient's body. Various medical tools for treatment and/or examination of the patient can be arranged in this shaft or the outer tube. For example, in the case of a resectoscope for high-frequency surgery, an electrode arranged at the distal end of an electrode carrier and subjected to high-frequency alternating current can be inserted into the shaft tube. For treatments to be performed on patients, such as the cutting of diseased tissue, the electrode carrier with the electrode is arranged on the resectoscope in such a way as to be movable relative to the shaft tube.

The electrode carrier is furthermore coupled with its proximal end, preferably movably, to a working element of the resectoscope, by which it can be moved along a shaft axis in order to carry out the cutting movement of the electrode. The working element is usually connected releasably to the outer tube. It has a carriage or contact body which is mounted so as to be movable longitudinally and to which the electrode carrier is coupled for joint longitudinal movement. The actuation or longitudinal displacement of the carriage relative to the working element is usually carried out by an operating surgeon. For this purpose, the operating surgeon grasps two handles or handle parts which are moveable toward each other and which are assigned to different assemblies of the resectoscope. In this case, according to the prior art, the movement of the working element or of the electrode carrier takes place, depending on the design of the working element, against or with the spring force of a spring which, in the case of the working element of the type in question, is usually formed as a leaf spring or as a leg spring and is arranged between the handles.

Since the aforementioned procedures take place inside the body, the operating surgeon is denied a direct view of the operation site. Therefore, a resectoscope of the type in question has an optical unit or a lens unit which is integrated into the resectoscope or the shaft. Such optical units usually consist of one or more lenses or a light guide, which is arranged within the elongate shaft of the resectoscope. The at least one lens transmits an image from the inside of the body through the shaft to the proximal end of the resectoscope. This transmission of the image information or of the light is effected either by several rod lenses arranged one behind another, which are arranged axially in the shaft, via electronic sensor means, such as a CCD chip, or via a light guide, such as a fiber optic, which is likewise arranged in the shaft of the endoscope.

In the resectoscope claimed here, the shaft has an outer tube and an inner tube, wherein a diameter of the inner tube is smaller than a diameter of the outer tube, so that the inner tube can be mounted in the outer tube. In the inner tube, the optical unit is routed to the distal end of the shaft.

In order for the operating surgeon to have a clear view of the operation site via the optical unit during the treatment, a flushing fluid is guided through the shaft into the interior of the body during the treatment. This flushing fluid can, for example, flush away tissue fragments that are released during the resectoscopy. Furthermore, the flushing fluid is used to remove cloudiness, caused for example by blood, from the field of vision of the optical unit. While the flushing fluid is usually supplied to the interior of the body via the inner tube through an inflow, the contaminated flushing fluid is aspirated through an outflow. This outflow is usually formed by an annular space between the outer tube and the inner tube. The proximal end of the resectoscope or of the shaft can be assigned a flushing device or a pump, so that the flushing fluid can first be guided into the body with a predeterminable pressure and, if necessary, can be removed again through the annular space by a slight vacuum.

For good viewing conditions during the operation, it is essential that the flushing fluid entering the body cavity forms a laminar flow, which runs at least almost parallel to a longitudinal axis of the shaft or an optical axis of the optical unit and allows visual monitoring of the electrode. As soon as the flow of flushing fluid is not laminar or even becomes turbulent, the visibility through the optical unit can become so poor that an operation cannot be carried out. For the formation of a laminar flow, it is particularly important that the flushing fluid within the inner tube can flow free of disturbance over the greatest possible distance. In known resectoscopes, such disturbance is caused by the electrode carrier, which is routed through the inner tube. In particular, eddies occur on the tubular carriers of the electrode and can lead to a turbulent flow and ultimately to poor visibility in front of the distal end of the resectoscope.

A further disadvantage of known resectoscopes is that the volume flow through the annular space between inner tube and outer tube is very limited on account of the high frictional forces of the flushing fluid on the inner wall of the outer tube and the outer wall of the inner tube. In order to increase this volume flow and in particular the discharge of the flushing fluid, it is obvious to reduce the diameter of the inner tube. However, this would reduce the space available for the instruments to be guided through the inner tube, in particular the optical unit. In addition, a reduction in the cross section of the inner tube would have a negative effect on the volume flow rate of the flushing fluid.

Proceeding from this, the problem addressed by the present invention is to make available a resectoscope in which the volume flows for the flushing fluid are optimized and at the same time a particularly laminar flow is formed in front of the distal end of the resectoscope, in order to improve the visibility during treatment.

A resectoscope for solving this problem has the features of claim 1. Accordingly, provision is made that the resectoscope has a shaft, at the distal end of which an electrode is arranged for manipulating the body tissue of a patient. This shaft is formed by an outer tube in which an inner tube is arranged. In addition, the resectoscope has a working element with a main body, wherein the inner tube is attached with a proximal end to the main body. At least one optical unit can be implemented or arranged through the inner tube. In addition, the inner tube serves as an inflow, and a volume between the outer tube and the interior serves as an outflow for a flushing fluid. The inner tube has different cross sections at its distal and proximal regions. Neither the cross section of the distal region nor the cross section of the proximal region is circular. This non-circular cross-sectional shape of the inner tube leads to an increase in the volume between the inner tube and the outer tube, which in turn has a positive effect on the flow resistance of the flushing fluid. In addition, this cross-sectional shape allows an instrument, such as the optical unit, to be guided through the inner tube and at the same time makes it possible to achieve a laminar flow of the flushing fluid through the inner tube.

Preferably, the invention provides that a cross section of a central region of the inner tube between the proximal and the distal region along the inner tube continuously transitions from the cross-sectional shape of the proximal region to the cross-sectional shape of the distal region. This continuous transition of the proximal cross-sectional shape into the distal cross-sectional shape leads to a laminar flow behavior of the fluid. In addition, the electrode carrier with the electrode can be arranged on an outer wall of the inner tube particularly reliably and easily and can be moved back and forth along the shaft axis.

In particular, it is conceivable that the cross section of the distal and/or proximal region has a waist, by which the cross section is divided into two regions, namely an upper region, which preferably serves to receive the optical unit, and a lower region, which preferably serves to transport the flushing fluid. Whilst this waist has the effect that the interior of the inner tube is particularly suitable for receiving the optical unit and for transporting the flushing fluid, this shape also results in an increase in the volume between the inner tube and the outer tube. It is conceivable that the upper region is dimensioned exactly in such a way that it can accommodate the optical unit. The waist also fixes the optical unit in its position within the inner tube. The waist prevents the optical unit from slipping into the lower region. Similarly, the waist clearly defines the lower region as a flow channel for the flushing fluid or for receiving a further instrument, or separates it from the upper region. The thickness of the waist or the width of the waist can vary; for example, it is conceivable that the two flanks of the waist touch or at least almost touch each other.

In an exemplary embodiment of the invention, provision can be made that a cross-sectional area of the upper region of the inner tube is equal to, smaller than or larger than a cross-sectional area of the lower region. Depending on the type of resectoscope or the nature of the optical unit and, if necessary, of other instruments that are guided through the inner tube, different cross-sectional shapes are conceivable for the inner tube.

In a particular development of the invention, provision can be made that a cross-sectional area of the upper region is oval or elliptic, wherein a longer axis of symmetry of the cross-sectional area intersects a horizontal plane through the inner tube perpendicularly, such that an in particular sickle-shaped channel for the fluid forms between a circular optical unit and an upper portion of the wall of the inner tube. In addition, it is conceivable that the cross section of the upper region has further widenings in order to enlarge the free passage surface for the flushing fluid through the inner tube. Thus, it has been shown that the formation of the sickle-shaped channel leads to an improved flow behavior of the flushing fluid, which ultimately leads to a very laminar flow in front of the distal end of the resectoscope.

The invention further provides in particular that the cross section of the distal region of the inner tube has at least one undercut, preferably two opposite undercuts, for fixing an electrode carrier with an electrode. The tubular carriers of the electrode carrier can be coupled to these undercuts. They can be coupled, for example, by being pushed on or clicked on. In particular, electrode carriers which are fork-shaped or have at least mainly two parallel carrier tubes, can be coupled to the outer wall of the inner tube in a particularly space-saving manner, without disturbing the flow behavior of the flushing fluid. The mounting of the electrode carrier onto the inner tube or the working element is also particularly reliable and easy, since the electrode carrier can only be pushed onto the inner tube from the distal direction. By means of the undercuts, the electrode carrier is fixed in its axial orientation and yet can still moved to and fro in the axial direction.

Furthermore, it is conceivable according to the invention that the distal region of the inner tube, in particular a wall of the distal region, has at least one receptacle, in particular openings, locking means, projections or the like, for the releasable coupling of an electrically insulating attachment, in particular an insulating insert. This attachment prevents the electrode, which is subjected to a high-frequency alternating electrical voltage, from coming into contact with the outer tube. For this purpose, the attachment is assigned to the distal end of the inner tube in such a way that it is located between the electrode and the proximal end in the region of the outer tube. By virtue of the releasable coupling of the attachment to the inner tube, it can be easily and reliably dismantled or assembled for cleaning or maintenance purposes, for example.

In a possible exemplary embodiment of the invention, provision can be made that the length of the distal portion is 60 mm to 210 mm, preferably 90 mm to 190 mm, 90 mm to 120 mm or 160 mm to 190 mm. The length of the proximal region can be 24 mm to 200 mm, preferably 90 mm to 170 mm. The length of the central region is dimensioned accordingly. In addition, it should be noted that the regions mentioned can also have other lengths.

Preferably, it is further conceivable that the cross section of the proximal region of the inner tube has an upper region and a lower region, wherein the upper region is round, preferably circular, and the lower region has two opposite straight and parallel or concave cross-sectional flanks. These straight or concave cross-sectional flanks in the proximal region prove particularly advantageous for guiding the proximal ends of the electrode carrier to the main body of the working element. Since this cross-sectional shape is limited to the proximal region of the inner tube, this shape has no influence on the formation of the laminar flow in the distal region of the inner tube. With slightly concave flanges, a larger gap can also be created between the electrode carriers and the tube, without thereby restricting the flow cross section too much. In contrast to straight flanks, the concave flanks have a preferred direction when pressed into a main body. This means that they bend inward into the flow cross section upon compression, whereas the straight flanks can bend inward or outward at random. In addition to this keyhole-like shape of the cross section of the proximal region of the inner tube, it is also conceivable that this region has other shapes which likewise serve to bring the electrode carrier particularly advantageously to the main body and at the same time serve to transport the optical unit and the flushing fluid to the distal end.

It is further conceivable that the upper regions of the proximal and distal cross section have the same cross-sectional shape, and/or that the lower regions of the proximal and distal cross section have the same cross-sectional shape. By virtue of this flexible design of the cross-sectional shapes, different embodiments of the inner tube are conceivable for different applications.

In another preferred exemplary embodiment of the invention, provision can be made that a proximal end of the inner tube is firmly connected to the main body of the working element, preferably pressed and welded or plugged and welded or joined together. It is particularly conceivable that a proximal end of the inner tube has a press-in portion, with which the inner tube can be pressed together with the main body. One possible method for the pressing is that of hydroforming. The inner tube is guided with the proximal end or the press-in portion into the main body of the working element and is subjected to a high pressure. By application of the high pressure, the inner tube or the press-in portion expands and presses into the main body. A very stable connection between the inner tube and the main body can thus be produced in a simple and cost-effective manner. If the pressing in is carried out by hydroforming, it is possible to dispense with welding, since a positive fit is created between tube and main body (undercut in the main body is pressed radially into the tube).

For pressing the inner tube into the main body, provision can be made that the cross section of the press-in portion has an upper region and a lower region, wherein the upper region is round, preferably circular, and the lower region has two opposite concave cross-sectional flanks, which are connected to each other by a round arc. It has been shown that these concave cross-sectional flanks are particularly suitable for guiding the inner tube or the press-in portion into the main body and pressing it there. In addition, this cross-sectional shape affords the advantages already outlined above. According to the invention, it is conceivable that the length of this pressed-in portion is 5 mm to 20 mm, preferably 10 mm.

A preferred exemplary embodiment of the invention is explained in detail below with reference to the drawing, in which:

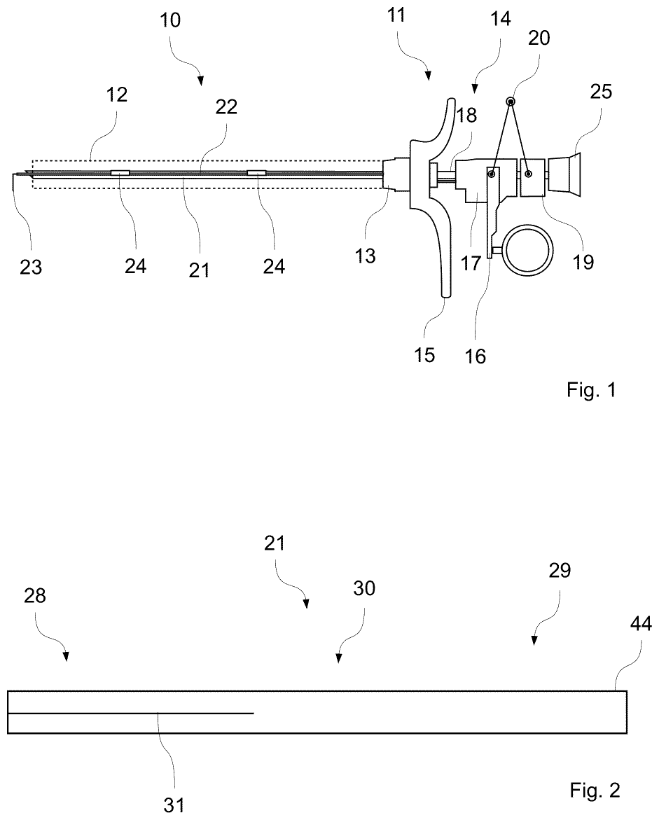

FIG. 1 shows a view of a resectoscope,

FIG. 2 shows schematic view of an inner tube,

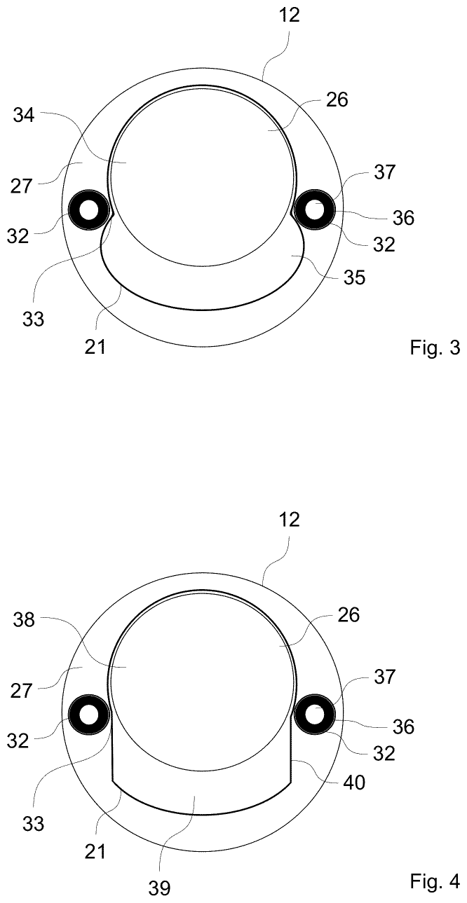

FIG. 3 shows a cross section of a distal region of the shaft,

FIG. 4 shows a cross section of a proximal region of the shaft,

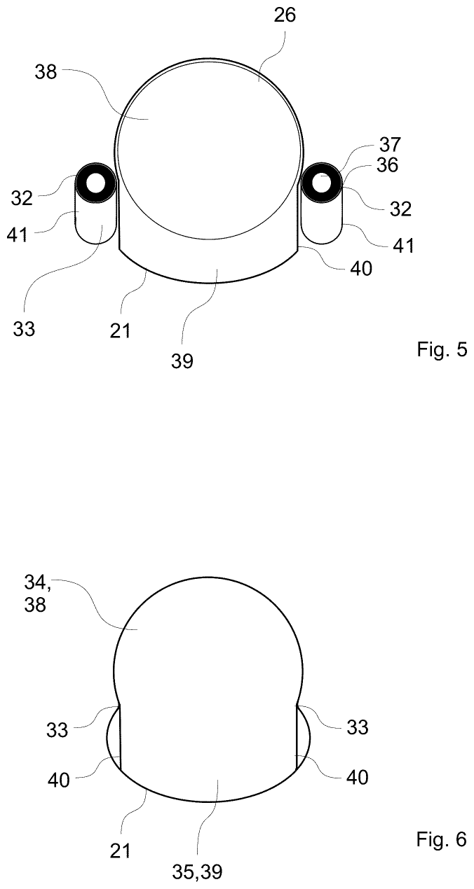

FIG. 5 shows a cross section of a proximal region of the shaft,

FIG. 6 shows a cross section of the inner tube, and

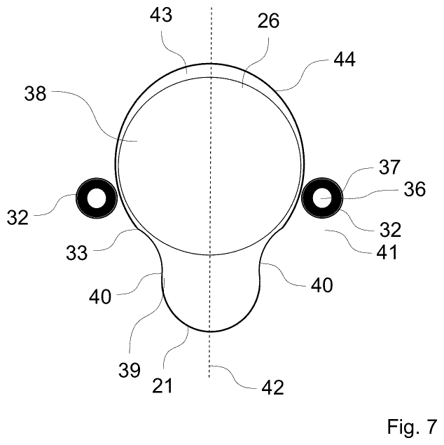

FIG. 7 shows a cross section of a press-in portion.

A possible exemplary embodiment of an electrosurgical handheld device, namely a resectoscope 10, is shown in a highly schematic form in FIG. 1. The resectoscope 10 has a working element 11 having an elongate, tubular shaft or an outer tube 12. This outer tube 12 is shown by hatching in FIG. 1 and can be fastened with a proximal end to a main body 13 of the working element 11.

The working element 11 has a handle unit 14 in addition to the main body 13. The working element 11 can have an in particular releasable handle 15. In the exemplary embodiment of the working element 11 shown here, a grip means 16 is assigned to a contact body 17. It is conceivable that the grip means 16 is screwed onto the contact body 17.

The contact body 17 is guided in a sliding movement on a tubular optical guide 18. Since the contact body 17 can be moved back and forth on the optical guide 18 along a longitudinal direction of the resectoscope 10 or a longitudinal axis of the outer tube 12, the contact body 17 is also referred to as a carriage. While the optical guide 18 is connectable with a distal end to the main body 13, an optical guide plate 19 is attached to a proximal end of the optical guide 18. The tubular optical guide 18 extends through the optical guide plate 19, so that the optical guide 18 is accessible from the proximal direction.

The grip means 16 and the contact body 17 are connected to the optical guide plate 19 via a spring element 20. This spring element 20 can be a tension spring or a compression spring, depending on the type of construction of the working element 11.

Starting from the main body 13, a tubular inner tube 21 extends in the distal direction. An electrode carrier 22 extends parallel to the inner tube 21. This electrode carrier 22 is guided through the main body 13 and is mechanically and releasably coupled with at least one proximal contact to the contact body 17. At a distal end, the electrode carrier 22 has an electrode 23. An electrical RF voltage can be applied to this electrode 23. By means of a thermal plasma that forms on the electrode 23, the diseased tissue can be manipulated or cut. To do this, the surgeon moves the grip means 16 relative to the working element 11. To stabilize the electrode carrier 22, it can be guided on the inner tube 21 through guides 24.

For performing the operation, a rod-like optical unit 26 is guided through the inner tube 21 or through the optical guide 18. A distal end (not shown here) of this optical unit 26 is directed toward the electrode 23, so that the surgeon has a view of the manipulation of the tissue. This optical unit 26 can be a rod lens system or a fiber optic. As is shown in FIG. 1, an eyepiece 25 or a camera is located at the proximal end of the optical unit 26.

The distal end of the inner tube 12 is assigned a releasable ring-like insulating attachment (not shown). This insulating attachment serves to electrically insulate the electrode 23 from the outer tube 12. In order for the insulating attachment to be electrically insulating, it can be made of plastic or ceramic.

To ensure that the surgeon's view of the operating site is not obstructed by blood or tissue during the operation, a flushing fluid can be applied to this site. For this purpose, the flushing fluid is passed through the inner tube 21 and exits, parallel to the optical unit 26, from the distal end of the inner tube 21. In order to remove the flushing fluid again from the interior of the body, it can be aspirated through a volume 27 between the inner tube 21 and the outer tube 12 by a suction device or pump (not shown). By generating a vacuum, the flushing fluid can be discharged via the volume 27.

FIG. 2 shows the inner tube 21 in a highly schematic form. This inner tube 21 has a distal region 28, a proximal region 29 and a central region 30. The core of the present invention is that a cross section of the distal region 28 of the inner tube 21 differs from a cross section of the proximal region 29. The cross sections transition continuously into one another in the central region 30. In addition, the inner tube 21, in particular the distal region 28 as shown in FIG. 2, has an undercut 31. This undercut 31, which is also formed on the opposite wall half of the inner tube 21, which wall half is not visible in FIG. 2, serves to receive the electrode carrier 22 with its guides 24. In this case, the electrode carrier 22 with the two tubes 32 and the guides 24 is pushed over the inner tube 21 in such a way that it is radially fixed in the undercut 31 and axially movable. Exemplary embodiments of the inner tube 21 are also conceivable in which the undercut 31 extends over the entire length of the inner tube 21.

FIG. 3 shows the cross section of the distal region 28 of the inner tube 21. Also shown is the cross section of the outer tube 12, of the optical unit 26, and of the two tubes 32 of the electrode carrier 22. It will be seen from this schematic illustration that the cross section of the distal region 28 of the inner tube 21 has a waist 33 or is waisted. This waist 33 separates the cross section into an upper region 34 and a lower region 35. As is already apparent from FIG. 3, the upper region 34 is circular, whereas the lower region 35 is oval. The circular upper region 34 is dimensioned exactly in such a way that it can accommodate the optical unit 26. The lower region 35 of the inner tube 21 is provided for allowing the flushing fluid to flow from the proximal region 29 to the electrode 23. On account of the available volume and the external placement of the electrode carrier 22, the flushing fluid can flow through the lower region 35 almost without interference, at least in the distal region 28 of the inner tube 21, so that the flow already behaves in a laminar fashion in the inner tube 21 and thus also outside the tube 21. The laminar flow is particularly advantageous for the visibility in front of the optical unit 26.

A further advantage of the waisted shape of the distal region 28 of the inner tube 21 is that the volume 27 between the outer tube 12 and the inner tube 21 is increased. In relation to a circular inner tube, the waist 33 creates space through which the flushing fluid can flow back to the proximal region 29. In addition, the tubes 32 of the electrode carrier 22 can be guided in this region. The electrode carrier 22 and the tubes 32 are shown here in section. The outer tube 32, the electrical insulation 36 and the electrical conductor 37 are thus visible.

As has already been mentioned, the cross section of the proximal region 29 of the inner tube 21 has a different shape. This shape is shown schematically in FIG. 4. It can be seen that the inner tube 21, in the proximal region 29, also has an upper region 38 and a central region 30. While the upper region 38 is formed in the same way as the upper region 34, namely serving to accommodate the optical unit 26, the lower region 39 has two straight and parallel cross-sectional flanks 40; alternatively, it is also conceivable that these flanks 40 are concave. The straight flanks 40 prove to be particularly advantageous for guiding the tubes 32 in the proximal region 29. There, the tubes 32 and the electrode carrier 22 describe a downwardly directed reflexed profile 41, as is indicated in FIG. 5. By this reflexed profile 41, the plane in which the electrode carrier 22 is located is moved, so that the proximal ends of the tubes 32 can be guided through the main body 13 of the working element 11.

FIG. 6 shows a cross section or a view over the entire length of the inner tube 21. It can be seen that the cross section of the distal region 28 transitions continuously over the central region 30 into the cross-sectional shape of the proximal region 29. The length of the regions 28 to 30 can differ for different embodiments of the resectoscope 10. For example, it is conceivable that that the length of the distal region 28 is 60 mm to 210 mm, preferably 90 mm to 190 mm, 90 mm to 120 mm or 160 mm to 190 mm, and the length of the proximal region 29 is 24 mm to 200 mm, preferably 90 mm to 170 mm. The length of the central portion 30 is dimensioned accordingly.

As an alternative to the straight cross-sectional flanks 40, it is also conceivable for these flanks to be concave. Such an exemplary embodiment is shown in FIG. 7. This concave design of the cross-sectional flanks 40 reduces the size of the lower region 39. Similarly, the volume 27 between the outer tube 12 and the inner tube 21 thus increases. A further advantage of this cross-sectional shape is the mounting of the inner tube 21 in the main body 13. For fastening the inner tube 21 in the main body 13, the proximal region 29 is first pushed into the main body 13. By virtue of the shape of the cross section shown in FIG. 7, the inner tube 21 can be fixed better in the main body 13. FIG. 7 also indicates a further advantageous feature, namely it is oval or elliptical. In this case, the oval or elliptical shape is oriented in such a way that a longer axis of symmetry 42 of the cross-sectional area intersects a horizontal plane through the inner tube 21 perpendicularly. Thus, a sickle-shaped channel 43 forms between the optical unit 26 and the undercut 31 of the inner tube 21. Flushing fluid can additionally flow through the inner tube 32 through this channel 43.

For fastening the inner tube 21 in the main body 13, provision is made that the proximal region 29 is connected to the main body 13. A particularly advantageous method for connecting the inner tube 21 to the main body 13 is hydroforming. In this case, a rear portion of the proximal region, the so-called press-in portion 44, which for example has the cross-sectional shape shown in FIG. 7, is subjected to a high pressure. This causes the wall of the inner tube 21 to be pressed into the opening of the main body 13. The components are then also welded together. In addition, further methods are conceivable for connecting the inner tube 21 to the main body 13.

### LIST OF REFERENCE SIGNS

10 resectoscope
11 working element
12 outer tube
13 main body
14 handle unit
15 handle
16 grip means
17 contact body
18 optical guide
19 optical guide plate
20 spring element
21 inner tube
22 electrode carrier
23 electrode
24 guide
25 eyepiece
26 optical unit
27 volume
28 distal region
29 proximal region
30 central region
31 undercut
32 tube
33 waist
34 upper region
35 lower region
36 insulation
37 conductor
38 upper region
39 lower region
40 cross-sectional flank
41 reflexed profile
42 axis of symmetry
43 channel
44 press-in portion

1. A resectoscope with an outer tube and an inner tube arranged in the outer tube and with a working element comprising a main body, wherein the inner tube is attached with a proximal end to the main body, wherein at least one optical unit is arranged in the inner tube, and the inner tube is formed as an inflow, and a volume between the outer tube and the inner tube is formed as an outflow for a flushing fluid, wherein that a cross section of a distal region of the inner tube differs from a cross section of a proximal region of the inner tube, neither the cross section of the distal region nor the cross section of the proximal region being circular.

2. The resectoscope as claimed in claim 1, wherein a cross section of a central region of the inner tube between the proximal and the distal region along the inner tube changes continuously from the cross-sectional shape of the proximal region to the cross-sectional shape of the distal region.

3. The resectoscope as claimed in claim 1, wherein the cross section of the distal and/or proximal region has a waist, by which the cross section is divided into two regions, namely an upper region and a lower region.

4. The resectoscope as claimed in claim 3, wherein a cross-sectional area of the upper region is equal to, smaller than or larger than a cross-sectional area of the lower region.

5. The resectoscope as claimed in claim 3, wherein a cross-sectional area of the upper region is oval or elliptic, wherein a longer axis of symmetry of the cross-sectional area intersects a horizontal plane through the inner tube perpendicularly, such that a channel for the flushing fluid forms between a circular optical unit and an upper portion of a wall of the inner tube.

6. The resectoscope as claimed in claim 1, wherein the cross section of the distal region of the inner tube has at least one undercut for fixing an electrode carrier with an electrode.

7. The resectoscope as claimed in claim 1, wherein the distal region of the inner tube has at least one receptacle, for the releasable coupling of an electrically insulating attachment.

8. The resectoscope as claimed in claim 1, wherein the length of the distal region is 60 mm to 210 mm.

9. The resectoscope as claimed in claim 1, wherein the cross section of the proximal region of the inner tube has an upper region and a lower region, wherein the upper region is round, and the lower region has two opposite straight and parallel or concave cross-sectional flanks.

10. The resectoscope as claimed in claim 1, wherein the length of the proximal region is 24 mm to 200 mm.

11. The resectoscope as claimed in claim 1, wherein the upper regions of the proximal and distal region have the same cross-sectional shape, and/or in that the lower regions of the proximal and distal region have the same cross-sectional shape.

12. The resectoscope as claimed in claim 1, wherein a proximal end of the inner tube is firmly connected to, or joined together with the main body of the working element.

13. The resectoscope as claimed in claim 1, wherein a proximal end of the inner tube has a press-in portion, with which the inner tube can be pressed into the main body.

14. The resectoscope as claimed in claim 13, wherein the cross section of the press-in portion has an upper region and a lower region, wherein the upper region is round, and the lower region has two opposite concave cross-sectional flanks.

15. The resectoscope as claimed in claim 13, wherein the length of the press-in portion is 5 mm to 20 mm.
